(19) United States
(12) Patent Application Publication
(10) Pub. No.: US 2010/0047181 A1
(43) Pub. Date: Feb. 25, 2010

(54) MIXTURE OF A VANILLOID RECEPTOR AGONIST AND A SUBSTANCE INHIBITING NERVE REGENERATION, USE THEREOF FOR PRODUCING A PAINKILLER, AND METHOD FOR APPLYING SAID PAINKILLER

(75) Inventor: Dominik Meyer, Zurich (CH)

Correspondence Address: RANKIN, HILL & CLARK LLP, 38210 Glenn Avenue, WILLOUGHBY, OH, 44094-7808, US

(73) Assignee: MESTEX AG, Zurich (CH)

(21) Appl. No.: 11/722,480

(22) PCT Filed: Dec. 22, 2004
(86) PCT No.: PCT/CH04/00750

§ 371 Date: Jun. 21, 2007

(51) Int. Cl. A61K 49/04; A61K 31/357; A61K 31/4355; A61K 31/437; A61K 31/352; A61P 19/02

(52) U.S. Cl. 424/9.41; 514/450; 514/291; 514/285; 514/456; 424/9.42; 424/9.4

(57) ABSTRACT

The following abstract will replace all prior versions of the abstract in the application:
The mixture comprises a vanilloid receptor agonist with a substance, which inhibits nerve regeneration. The mixture is suitable for use, especially, as a painkiller.

The invention relates to a mixture of a vanilloid receptor agonist, which is also referred to as “vanilloid” in the following, and a substance, which inhibits nerve regeneration and is also referred to as “inhibitor” below, their use for producing an agent, which is also referred to as a “substance combination” in the following, for treating pain and possible methods for the systemic, regional, topical or local ingestion or administration, or application/injection of these agents. Furthermore, the use is combined by means of a strictly systemic (peroral, transcutaneous injection or the like) or a local/regional (tramsmucosal, transcutaneous, injected) ingestion or administration, of two or more components.

The inventive mixture is also suitable for prolonging the action of vanilloid receptor agonists, especially for the type 1 vanilloid receptor (for example resiniferatoxin) for the long-lasting treatment of any form of pain, which is passed on by nociceptive fibers, especially also neurogenic pain. Furthermore, the substance combination can be used for prolonging any other effect of vanilloid receptor agonists, such as thermoregulation or other known effects, by inhibiting the regeneration of the nerves in question.

The use of this mixture is described below, by way of example, especially for the treatment of joint pain. However, said method is to be used for any form of pain. Pain, emanating from joints, frequently has its origin in the area of the joint capsule or in the area of a bone in the vicinity of a joint. In this connection, many analogies may come into consideration, such as arthrotic or arthritic forms of disease, irritation or injury to the disk structures of joints, infections, autoimmune processes, etc. In all cases, which are of interest within the scope of this invention, the resulting pain emanates from nociceptive nerve fibers in the region near the nerve. Nociceptive fibers are also referred to as C fibers or A delta fibers. If an analgesics substance (such as a local anesthetic, vanilloid receptor agonist or morphine) is injected into a joint so diseased, the symptoms of the patient are alleviated. However, the substances customary at the present time, act for only a limited time, so that the symptoms generally return.

Generally, the following methods are used at the present time for the treatment of painful, diseased joints:
physiotherapy/movement therapy
systemic analgesic/antiphlogistic therapy (etc.)
local analgesic/antiphlogistic methods (etc.)
surgical methods:
arthroscopic: debridement, joint toilette, etc.
open/mini-open: joint replacement, joint reinforcement, etc.

A series of known substances has also already been proposed in the literature for the treatment of painful, inflamed joints, especially:
the injection of capsaicins
osmic acid or radioactive substances, such as technetium 99, which lead to a synoviorthesis
injection of local anesthetics, hyaluronic preparations (etc.)
injection of antiphlogistic agents
injection of contrasting agents for joint diagnostics
the flushing of joints for a joint toilette
chemical, thermal, electrical or surgical ablation of joint-supplying nerves, at a site remote from the joint.

The known method of synoviorthesis has the disadvantage that molecular structures are destroyed and, in particular, proteins, which initiate inflammations in the arthritis process and partly also in the development or arthroses, are denatured. Moreover, a fibrosis of the joint capsule, which is less inflammatory and, accordingly, also less painful, develops. At the same time, due to the fibrosis of the joint, which occurs during the synoviorthesis, the hyperemia, which is generally present and also to be treated, is reduced, resulting in therapeutic benefit. However, the fibrotic scarring after synoviorthesis can lead to a decreased mobility of the joint as well as to a decreased production of synovial fluid and to the distraction of joint cartilage. This undesirable fibrosis of the joint capsules should be avoided and only the sensitive innervation of the joint should be eliminated.

The EP-B 0 998 288 of CAMPBELL discloses the use of capsaicin and analogues thereof together with a local anesthetic. Capsaicin produces a strongly burning sensation, which can be ameliorated only by a simultaneous or sequential administration of a local anesthetic. Local anesthetics have an antagonistic effect with regard to capsaicin, which decreases the effectiveness of the capsaicin, so that a larger dosage or a higher concentration of capsaicin becomes necessary, if the latter is to be used together with a local anesthetic. At the dosage, so required, capsaicin has the effect of causing an inflammatory reaction and, when used in a joint, causes damage to the cartilage. However, a combined use of the substance combination, claimed here, together with local anesthetics, for reducing the pain during the injection is entirely possible.

U.S. Pat. No. 6,326,020 of KOHANE ET AL discloses the use of a vanilloid receptor agonist together with a sodium channel blocker of the tetrodotoxin type, a “site 1 sodium channel blocker” for achieving a nerve block. This class of substances, however, only has an additional toxic effect on the nerves and does not prolong the action in the sense of this invention.

All previously used substances and methods lead to a relatively brief or incomplete freedom from pain or cause lasting damage to the joint.

The invention is to provide a remedy here. By mixing the vanilloid receptor agonist with a substance, which inhibits nerve regeneration, it is possible to reduce the necessary dosage of neurotoxic vanilloid receptor agonist clearly and, with that, also to diminish the side effects, such as a painful burning during the injection, local inflammation as well as damage to the tissue, such as cartilage damage at the joint. When a sensitive patient or a painful site is injected, a local anesthetic substance, such as lidocaine, can be injected (as suggested by Campbell et al.) either before, along with or after the injection of the substances claimed here. One way of reducing these symptoms and side effects further and of increasing the efficiency of the treatment consists therein that the release of one or both or several of the active substances is retarded by suitable pharmaceutical formulations.

Pursuant to the invention, this objective is accomplished with a mixture of the distinguishing features of claim 1, with the use of such a mixture having the distinguishing features of claim 40 as well as with a method for treating joint pain or other pain having the distinguishing features of claim 48.

The effect of vanilloid receptor agonists (as analgesics, brought into the body locally or systemically, topically or in any other way) is reversible by healing the nerves, especially the C-delta and A-delta fibers. Surprisingly, the effect of the analgesic is increased and/or prolonged by the addition of substances, which inhibit nerve regeneration (locally or systemically, topically or in the body).

The realization that vanilloid receptor agonists damage receptor-carrying nerves, so that they no longer can pass on a response, is an essential feature of the invention. As a result, the selectively damaged nerves (such as a C fiber or a C fiber end) becomes more sensitive to substances, which inhibit nerve regeneration and affect nerve homeostasis by blockade of the tubular or axonal transport system, by tubulus aggregation or aggregation inhibition, tubulus polymerization or tubulus depolymerization, tubulus damage due to other mechanisms affecting the neurotubuli or tubulin, or by tubular or axonal, retrograde “suicide transport”. Not only is nerve regeneration inhibited by these means, but the nerve is also partly deadened and a breakdown of the nerve is induced, as a result of which regrowth, new growth, regeneration or sprouting out no longer is possible or is reduced clearly.

It is a significant aspect of the invention that the, substance which inhibits nerve regeneration, can penetrate into the nerve surprisingly already with very little damage to the sensitive nerve fiber by the vanilloid receptor agonist and thus bring about an effect in the sense of the vanilloid receptor agonist, in a dosage or concentration, which, for either of the two substances alone, would not have had the desired effect. Due to the selective action of the vanilloid receptor agonist, the nerve is damaged so that the substance, which inhibits regeneration, can already develop its effect, while other fibers, not sensitive to vanilloids, remain unaffected. It is therefore particularly efficient to apply or inject the two substances as a mixture, since the nerves can be damaged locally in this way and, at the same time, the substances can also damage the same nerves, nerve ends or ganglia or nerve cells, which are to be treated, in the desired manner over a period of time. This therapy has been developed primarily for pain therapy. Since not all substances, which inhibit nerve regeneration, attack the neuronal tubuli at the same place, it has proven to be advantageous to mix two or more different tubulus poisons, such as Taxol and Vincristin. Furthermore, a nerve, which still functions partially, cannot transport new sensitive receptors to the nerve end and therefore remains not sensitive.

As already mentioned above, it is also conceivable to apply the combination of two (or more) substances systemically, to apply only one substance systemically and the other substance locally (also topically on the skin or the mucous membrane) or regionally, simultaneously or temporally offset (in the latter case, the vanilloid receptor agonist should preferably be used first and the substance, which inhibits regeneration, is used next or with delay. Furthermore, the preparation of one or both or several of the components of the mixture with or as temporally delayed or controlled-release pharmacological preparations or carrier media is possible and meaningful.

The following are the advantages achievable with the invention:
The local administration of vanilloid receptor agonists and/or inhibitors leads mainly to a local effect and permits systemic and regional side effects to be reduced and also, in particular, the amount of substances required to be decreased.
The combination of the two substances has a synergistic effect and improves the efficiency and duration of action of the two individual substances.
The local administration of vanilloid receptor agonists can reach the nerves, which are to be treated, locally; the systemic administration of the substance, which inhibits regeneration, achieves that the corresponding nerve is exposed to the inhibition over the whole of its length, which may have a favorable effect on prolonging and intensifying the action. Local side effects can be reduced by the systemic administration.
The systemic administration of vanilloid receptor agonists and analogs, especially of newer substances, which can be administered orally, has the advantage that a selective application is not necessary and local side effects are reduced and that the nerve structures affected can be reached over their whole length. The additional local inhibition by means of substances, which inhibit regeneration, prevents local regeneration.
The systemic administration of vanilloid receptor agonists and analogs, especially of newer substances, which can be administered orally, together with substances, which inhibit regeneration, has the advantage that a selective administration is not necessary and that local side effects are reduced. All substances may, however, also be administered together or separately, intravenously, intra-arterially, interperitoneally, subcutaneously, percutaneously (also mucous membranes and the epithelium of the urinary tract), perorally, inhaled or the like. The advantage of the systemic administration is that the nerve structures affected can be reached over their whole length and several pain localizations can be treated simultaneously. The additional local inhibition by means of substances, which inhibit regeneration, may optionally be administered and prevents local regeneration; alternatively or additionally, a vanilloid receptor agonists can also be used locally.
The method can be carried out by people, who are not specialists.
The method can be carried out with a thin needle, even with a non-arthroscopic needle.
The method does not run the risk of developing an infection, in contrast to a popular method of injecting cortisone, which strongly promotes infections locally, since cortisone inhibits the immune system locally.
The method leads to a sensitive denervation, that is, to a switching off of pain-conducting nerves.
Expansion of the joint mobility by eliminating painful movement limitation in contrast to synoviorthosis, which results in movement limitation due to the capsule fibrosis, which develops.
Positive preparation for a later arthroplasty, when administered at a joint. Due to the stimulating effect of the stressing without pain, which then takes place once again, the bone formation in the extremity, especially in the vicinity of the joint, is promoted and bone in the vicinity of the joint develops a structure, which is advantageous for holding a prosthesis later on.
No local fatty tissue absorption (lipolysis).
No weakening of collagenous tendon, ligament or capsule structures.
Due to the highly selective action of the vanilloid receptor agonists, there is no myokinetic impairment, no matter where the substance was injected.
Due to the highly selective action of the vanilloid receptor agonists, there is no proprioceptive impairment, no matter where the substance was injected.

The invention is described in the following for use in man, the dosages given accordingly referring to human administration. However, the invention is also suitable for the veterinary area or for laboratory experiments, the dosage having to be adapted to the bodyweight of the respective animal.

### Intraarticular Application:

For this application, the joint capsule is used to concentrate the effect of the claimed substance combination at the place, where the pain develops, and, by these means, to permit a concentration of one or both of the components, which is higher locally than that possible without the protective joint capsule and, at the same time, to have relatively little effect on vessel and nerve structures and other structures in the vicinity of the joint. Accordingly, a long-term alleviation of the feeling of pain, emanating from the diseased ligament-capsule-joint complex, is achieved by inhibiting or switching off conduction. This method can be used preventively or therapeutically.

In a preferred embodiment, an x-ray contrasting agent, such as a barium salt, or an MRI contrasting agent is used in addition to the substance combination so that the distribution of the substance combination in the intracapsular space can be checked visually.

Depending on the method, the following substances may be used as contrasting agent:
X-ray, CT: Iodine-containing substances, such as triodinated benzoates or iopamidol, ideally 30-80 g/100 mL or, for example, 10% of a different contrasting agent, such as barium
MRI: For example, gadolinium, for example, 469.01 mg of gadopentate dimeglumide, 0.99 mg of meglumin, 0.4 mg of dimethylenetriamine pentaacetate per 1 mL.

For a further embodiment, an antibiotic, disinfecting and/or sterilizing substance is additionally added to the substance combination.

For a further embodiment, a viscous additive, such as glycosmainoglycan, chondroitin sulfate and/or hyaluronic acid and comparable substances, preferably in a concentration of 0.1-50 mg/milliliter of injections solution, are used in addition to the substance combination. This leads to an improvement in the mechanical sliding of the joint and decreases pain during the injection. Furthermore, it was found that the action is improved and prolonged.

For a further embodiment, a vasoconstrictor, preferably adrenaline, noradrenaline or other, similar, preferably alpha-adrenergic vasoconstrictors are used in addition to the substance combination. With adrenaline, the total dose of active substances can be increased by the factor of approximately 1.5 to 5, since the systemic action is reduced by the decreased absorption. The adrenaline concentration may amount to 1:10,000 to 1 80,000 to 1 200,000. The total dose of adrenaline is less than 0.25 mg. A 50 mL solution of 1:200,000 adrenaline contains 0.25 mg of adrenaline.

For a further embodiment of the invention, a substance with antiphlogistic activity, for example, a nonsteroidal antirheumatic agent, such as a COX-2 inhibitor, acetylsalicylic acid, etc. is used in addition to the substance combination.

For a further embodiment, a local anesthetic is used in addition to the substance combination or one of the components thereof in order
to anesthetize the puncture channel
to anesthetize the region of the middle application
to anesthetize the nerve, which is to be closed down
to anesthetize the spinal marrow
to anesthetize the surface (skin or mucosa or epithelium of the urinary tract or intestine), on which the substance or substances is/are to be applied.

The local anesthetic or components of the substance combination or combinations used can be released immediately or, in a suitable pharmaceutical form, with delay.

For a further embodiment, a steroid is used in addition to the combination of substances, in order to control any inflammatory reaction, which may occur. With this, moreover, a causal treatment of painful, inflammatory joint diseases, which supports the symptomatic, neurolytic treatment, can be added more readily. Betamethasone has proven to be particularly suitable, for example, in the form of 5 mg of betamethasone as dipropionate (crystalline suspension) and 2 mg of betamethasone as disodium phosphate (solution in 1 mL can be added to the amount that is to be injected). This solution is equivalent to 45/23 mg of prednisone/prednisolone.

For a further embodiment, glycerin is used as solvent in addition to the substance combination. Glycerin also has neurotoxic properties (especially, however, if it is injected intraneurally). Moreover, glycerin can lubricate the joint, so that there is also a physical effect here. The concentration of glycerin preferably is between 10 and 95%.

For a further embodiment, calcium Ca²⁺ or comparable ions are used in addition to the combination of substances in the solvent at a concentration higher than the physiological concentration and released simultaneously or with delay. Calcium is necessary for the action of the vanilloid receptor agonists and improves their action when present in a hyperphysiological concentration. The concentration of calcium preferably is greater than 2 mmolar and especially greater than 4 mmolar.

For a further embodiment, a change in the pH is produced at the site of action, preferably by mixing the vanilloid receptor agonists with a suitable, buffered medium. An alternate activity profile can be produced by shifting the pH. The action of the vanilloid receptor agonists is intensified at a pH below 7.4 and the painfulness of the injection is clearly reduced at a pH above 7.4.

For a further embodiment, therefore, the pH at first is adjusted to a value higher than 7.4 by the application or injection of suitable buffer media, which can also be released with delay by microencapsulation or in solid form, for example, as a powder or as an implant, such as a bone-replacement material. Subsequently, the pH drops, preferably within minutes to hours, to a value below 7.4. Instead of glycerin, water, salt solution, sodium iothalamate, iophenylate, ricin, polyethylene glycol or polypropylene glycol can be used as solvent. As solvent, glycerin has the advantage that it is hyperbaric and also already somewhat neurotoxic.

Some materials, such as calcium (particularly at a concentration of more than 2 mmolar and preferably of more than 4 mmolar, magnesium, antioxidants, preservatives and excipients, especially sodium bisulfite at a concentration of more than 0.2%, HaHSO₃, ammonium compounds, such as ammonium sulfate (NH₄)₂SO₄, 2-10 (−30%), polysorbate 80 (PS80) 0.025 mg/milliliter, have proven to intensify the action of the combination of substances.

The combination of substances preferably is injected dissolved in a solvent, which is compatible with the body, and advisably is injected in a volume, which corresponds to the available space in the joint that is to be treated, so that this space is filled barely to firmly. With that, the advantage of an optimum local distribution of the substance combination is achieved. It is, however, also possible to inject less liquid. In that case, however, the joint must be moved well in order to improve the distribution of the substance combination.

The liquid volume, to be injected into the intracapsular region, may vary from 0.1 to 150 mL. For a finger joint, a maximum of about 1 mL is sufficient, for the shoulder joint, a maximum of 10 mL, for the knee joint, a maximum of 30-50 mL and preferably of not more than 2 mL.

The dosage of the combination of substances depends on the localization and indication.

### Systemic Administration

When the combination of substances or parts thereof is administered orally, an antiemetic is used as well. For a preferred administration of the substance combination, a substance, such as a proton pump inhibitor, which does not put too much strain on the stomach, is used as well for the systemic oral administration.

### Topical Administration

For a further embodiment, the inventive mixture is applied with a suitable topical patch or plaster.

For a further embodiment, the inventive mixture is administered as a gel, ointment, cream, tincture or the like, optionally together with a permeation-promoting substance, such as ethoxylated ethylene diglycol, purified phosphatidyl choline, propylene glycol dipelargonate (DPPG) or with glycosylated, ethoxylated glycerides.

For a further preferred embodiment of the invention, one or both parts of the substance combination are transported through the skin on mucous membrane by means of iontophoresis or microinjection (air or hydraulically or as a powder) or similar methods with suitable, conventional media.

### Regional Administration

For a further form of administration, the spinal marrow or a peripheral nerve or nerve plexus is treated in the innervation area or locally with the substance combination or a part thereof for the treatment of pain.

### Intraoperative or Postoperative Administration

For a further administration, the surgical area or parts thereof or a wound, explored by endoscopy or arthroscopy, is flushed with the substance combination or the latter is dripped thereon or the substance combination is applied as a powder, a paste, in wax or as a gel or deposited topically in a similar form. Post-operative pain is reduced or prevented in this manner.

### Special Embodiments

For a special embodiment, the substance, inhibiting nerve regeneration, is a cytostatic agent. It may also, however, be an antimycotic agent or an antibiotic or an analog thereof. The substance, inhibiting nerve regeneration, may also be a neurotoxin.

For a special embodiment, the substance, inhibiting nerve regeneration, is selected from that group of materials, which interfere with the tubular system and thus inhibit the tubular or axoplasmatic transport of molecules of all types or utilized for the suicide transport. By these means, it is prevented that the nerve, after being damaged by the vanilloid receptor agonist, cannot regenerate, because the necessary tubular transport of proteins and other building blocks is interrupted.

For a further embodiment, the substance, inhibiting nose regeneration, may also be a tubulus poison. The tubulus poison may bond to tubulin, preferably neuronal tubulin, or to microtubuli. The tubulus poison, bound to tubulin or microtubuli, stabilizes the tubulin and the microtubuli against the polymerization and, in so doing, prevents them from functioning. By these means, it is prevented that the nerve, after being damaged by the vanilloid receptor agonist, can regenerate, because the necessary tubular transport of proteins and other building blocks is interrupted.

For a further embodiment, the vanilloid receptor agonist is one for the vanilloid receptor type 1 (TRPV1). Selective damage to pain fibers is achieved by these means.

For a further embodiment, the vanilloid receptor agonist is selected from the following substances: resinifera compounds, of which, in particular, the resiniferatoxin (RTX), olvanil, capsiate, civamide, SDZ-249-665, DA-5016, arvanil, scutigeral, isovelleral, phorbol 12,13-didecanoate 20 homovanillate, phorbol 12,13-dinonanoate 20 homovanillate, tinyatoxin and comparable substances, as well as analogs, derivatives and salts of the compounds mentioned above.

For a further embodiment, the vanilloid receptor agonist is a vanilloid, especially from the group of trans-8-methyl-N-vanillyl-6-nonenamides, N-vanillyl-nonamides, beta-aminoethyl-substituted phenylalkanamides, methylene substituted N-phenylmethylalkanamides, N-((substituted phenyl)methyl)-cis-monounsaturated alkenamides, beta-aminoethyl-substituted phenyl compounds, N-((substiotuted phenyl)methyl diunsaturated amides, N-oleoyidopamine, a capsaicin analog, such as transcapsaicin, dihydrocapsaicin, cis-capsaicin, as well as analogs, derivatives and salts of the aforementioned compounds. Selective damage of the pain fibers is achieved by these substances.

For further, special embodiment, the substance, inhibiting nerve regeneration, is a tubulus poison or a cytostatic agent. The substances are neurotoxic per se and bring about, in particular, a retardation of nerve regeneration.

In this connection, the cytostatic agent is selected advantageously from the group of vinca alkaloids, preferably Vincristin, Vincristin sulfate, Vinorelbin or Vinflunin. The cytostatic agent may also be selected from the group of Taxoids/Taxols, such as Paclitaxel, Nosacapines, especially brominated Noscapines (for example, 5-bromonoscapin, and reduced 5-bromonoscapin) and analogs, as well as Phenytoin.

The tubulus poison may be selected from the group of vinca alkaloids, preferably the Vincristin, Vincristin sulfate, Vinorelbin or Vinflunin. These substances are particularly efficient in damaging nerve regeneration. For a further embodiment, the tubulus poison is selected from the group comprising Ansamitocin P-3 (Maytansinoid), Phomopsin A, Dolastatin 10, Ustoloxines; Arenastatin A, tricyclic pyrones (such as 3-pyridyl benzopyran), Rhizoxin and analogs, all from the group of colchicines or colchicine-like substances. These substances have the advantage that they are tolerated well.

The tubulus poison may also be selected from the group of podophyllotixins, combretastasins and nocodazols, from the group of coumarins or dicomarols or from the group of quinolones, particularly ciprofloxacin, cinoxacin, enoxacin, fleroxacin or from the group of sulfonamides, particularly sulfamethoxazol, or from the group of flavonoids, particularly quercetin, indolyloxazolin derivatives, pyrimidinyl pyrazolates and analogs.

For a further embodiment, the tubulus poison is selected from the group of Taxoids/Taxols, Paclitaxel, Nosacapins and analogs, podophyllotoxin, combretastasin, nocodazols, griseofulvin, phenytoin and analogs. These substances have the advantage that they are tolerated well. Noscapins have very slight side effects and can also be readily taken by mouth.

For a further embodiment, the tubulus poison is selected from the group of coumarins, dicoumarol, quinolones, sulfonamides, quercetin, indolyloxazolin derivatives and pyrimidinyl pyrazolates. The coumarins have very few side effects aside from producing, at times, an increased tendency to bleed.

For a further embodiment, the substance, inhibiting nerve regeneration, is selected from the group of semaphorins, preferably semaphorin III. The semaphorins inhibit regrowth of nerves locally.

For a further embodiment, the substance, inhibiting regeneration, is doxorubicin or a lectin, such as ricin, abrin, volkensin, modeccin and preferably saporin or an analog thereof. These substances have the advantage that, in part, they are extremely toxic. The nerves in the nucleolus are damaged by the tubular and axonal suicide transport and nerve regeneration is prevented so extremely efficiently, that this is an advantage of this application.

For one embodiment, the antimycotic agent can be selected from the group of griseofulvins and analogous antimycotic agents or from the group of coumarins or dicoumarols.

For a further embodiment, the mixture, in addition, contains a local anesthetic. As a result, there is less pain during the injection. At a suitably high concentration, the local anesthetics themselves are additionally neurotoxic and support the effect desired.

For a further embodiment, the mixture additionally contains an x-ray contrasting agent, preferably in the form of gadolinium-containing, iodine containing or barium-containing substances. By these means, the distribution in the body can be documented accurately.

For a further embodiment, the mixture additionally contains a spheroid information reactions can be suppressed by this mixture and a synergistic effect can be achieved with respect to joint pain.

For a further embodiment, the mixture additionally contains a vasoconstrictor, preferably adrenaline, noradrenaline, phenylephrine or ornipressin. By these means, a lesser systemic distribution and, with that, a better systemic compatibility and better locum effectiveness can be achieved.

For a further embodiment, the mixture is dissolved in a solvent, with which the body is compatible, preferably, a pharmacologically acceptable vehicle, especially from the group of sodium chloride injections solution, Ringer's injection solution, isotonic dextrose, sterile water, dextrose solution, lactated Ringer's injections solution or mixtures thereof. This is essential if the substance mixture is to be injected or if the body region is to be flushed with the substance mixture.

For a further embodiment, the mixture additionally contains a permeation promoter, preferably dimethyl sulfoxide, ethoxyethylene diglycol, ethanol, phosphatidyl choline, propylene glycol dipelargonate (DPPG), or glycosylated ethoxylated glycerides. This is of advantage especially for topical application on the skin, but also to improve the permeation in tissues and through mucous membranes.

For a further embodiment, the mixture additionally contains a calcium salt, by means of which the effectiveness of the vanilloid receptor agonist is improved, since the toxicity is based partly on increasing the intracellular Ca²⁺ level. Advisably, the calcium ion concentration is greater than 2 mmolar and preferably greater than 4 mmolar. By these means, the effectiveness of the vanilloid receptor agonist is improved, since the toxicity is based partly on increasing the intracellular Ca⁺² level. The same effect can also be achieved if the sodium level in the solution is increased selectively or if the total ion concentration in the solvent or portions thereof of is greater than the physiological concentration.

For a further embodiment, the mixture additionally contains a glycosaminoglycan (chondroitin sulfate) its derivatives or salts. The glycosaminoglycan advisably constitutes 0.5% to 10% and preferably 1.0% to 3% of the total mixture. Due to the chondro-protective effect of the glycosaminoglycan, the burning sensation during the injection is suppressed and the joint is taken care of. A partial bonding of the vanilloid receptor agonist to the glycosaminoglycan may bring about a delayed release over a longer period of time.

For a further embodiment, the mixture additionally contains hyaluronic acid, its derivatives or salts. Advisably, hyaluronic acid constitutes 0.1% to 10% and preferably 0.5% to 3% of the total mixture.

For a further embodiment, the mixture is dissolved in a buffer solution with a pH above 7.6 and preferably above 8.5. By these means, the burning painfulness during the injection is decreased, since the ion channels of the receptor are opened later.

For a different embodiment, the mixture is dissolved in a buffer solution with a pH below 7.2 and preferably below 6.0. By these means, the effect of the vanilloid receptor agonist is increased, since the receptor ion channels open more easily and earlier.

For a further embodiment, the mixture is formulated in a suitable pharmaceutical preparation, which permits a retarded release of the mixture. By these means, the effect can be optimized with fewer side effects.

For a further embodiment, the mixture contains a combination of several substances, which inhibit nerve regeneration. By these means, nerve regeneration is inhibited even more efficiently and with fewer side effects than it would be with only one substance.

For a further embodiment, the mixture contains a concentration of several vanilloid receptor agonists. Since not all vanilloid receptor agonist dock at or affect the same receptors in the same manner and since they do not all have the same side effect profile, mixtures of such vanilloids may develop advantageous synergies.

The inventive mixtures may be used for producing an agent for treating sensations, which are passed on by nerves, which carry vanilloid receptors. Such nerves are damaged highly selectively by vanilloid receptor agonists.

For a variation, the inventive agent may be used for producing an agent for the local treatment of sensations, which are passed on by nerves, which carry a vanilloid receptors. Such nerves are damaged highly selectively by vanilloid receptor agonists.

The inventive agent is intended for the treatment of the following indications:
a) wound pain after surgery in the form of a flushing solution for intraoperative application for an open or arthroscopic or endoscopic surgery, including liposuction;
b) joint pain by intraarticular injection in the case of

arthrosis

rheumatoid arthritis

infectious arthritis

chondrocalcinosis

ligamentary damage

meniscus lesion

cartilage damage

synovitis

arthrofibrosis

Sudeck's disease

necrosis of portions of a joint

neuropathic joint pain
c) bone pain after bone surgery by application on the bone after iliac crest osteotomy or Hallux-Valgus correction
d) bone pain by injection into the bone in the case of necrosis of the head of the femur or into the body of a vertebra in the case of osteocondrosis;
e) joint stiffness, especially in the case of arthrofibrosis or a frozen shoulder;
f) muscle pain due to intramuscular injection, especially if there is a tear in muscle fibers, if there is pain after muscular exertion or in the case of spastic diseases;
g) painful meniscus, if there is degeneration of or a tear in the meniscus;
h) treatment of back pain by injection into the intervertebral disk in the case of the degeneration of or a tear in the intervertebral disk;
i) painful nerves, especially trigeminus neuralgia, neurinoma, Morton neurinoma, phantom pain or scar neurinoma;
j) toothache, especially in the case of dental caries, all forms of toothache, before, during or after tooth extraction, before, during or after a tooth implanting, applied topically in the case of parodontitis, or applied topically in the case of an exposed neck of a tooth;
k) pleuritic complaints
l) intestinal complaints, especially in the case of ulcerous colitis, Crohn's disease, anal fissures or hemorrhoids.

For a special form of a use, the vanilloid receptor agonist has a concentration or dosage locally, which is equivalent to the following parameters:
a) In the case of resiniferatoxin as vanilloid receptor agonists, a concentration of 5 nmolar to 600 μmolar and preferably of 100 to 5000 nmolar or a dose of 1 ng to 15 mg/kg of body weight and preferably of 10 ng to 50 μg/kg of body weight.
b) In the case of capsaicin as vanilloid receptor agonist, a concentration of 0.05% to 10% weight of the total mixture or a dose of 0.1 to 200 mg/kg of body weight.

In the case of a retarded release, for example, in the form of microencapsulation, the concentration in the capsules or in the mixture to be applied is correspondingly higher in order to attain the desired local concentration.

For a special form of use, the substance, inhibiting nerve regeneration is used at a dosage of 0.0001 mg to 50 mg for an intraarticular application. Preferably, the dosage is 0.001 mg to 1 mg.

For a different form of use, the substance, inhibiting nerve regeneration, is used in a single dosage or in repeated dosages of initially 0.001 to 600 mg for applications per os.

For a special form of use, the agent dissolved in a suitable solvent, which is compatible with the body is injected locally into the pain-affected tissue structure or is applied dropwise on a surgical wound or applied at a peripheral nerve or ganglion or administered transcutaneously.

The inventive agent can be used for a process of treating joint pain, in that it is injected locally into the intracapsular region or into the joint capsule of the joint affected by pain. Advisably, the agent is dissolved in a solvent, which is compatible with the body. Preferably a volume of 0.1 to 150 mL of the solution is injected locally into the intracapsular region or into the joint capsule of the joint affected by pain. By these means, the nociceptive nerve fibers become insensitive to pain for at least 14 days and preferably at least 8 weeks. The agent advisably is used at such a concentration, that neurolysis occurs.

The agent may be used locally, regionally, systemically (intravenous, peroral, subcutaneous, intramuscular, etc.) or topically on the skin or mucous membranes.

Preferably, the vanilloid receptor agonist and the substance inhibiting nerve regeneration are used simultaneously. By these means, good local control and few side effects can be achieved. In particular, a good local and temporally synergistic effect of the two substances is achieved.

Alternatively, the vanilloid receptor agonists can be used first and, after that, the substance inhibiting nerve regeneration. By these means, the vulnerable phase of nerve regeneration can be utilized better in some situations and a more efficient effect can be achieved than in the case of a simultaneous application. Alternatively, the substance, inhibiting nerve regeneration, can also be used first and, after that, the vanilloid receptor agonists. By these means, the vulnerable phase of nerve regeneration can be utilized better in some situations and a more efficient effect can be achieved.

The vanilloid receptor agonist and/or the substance inhibiting nerve regeneration can also be used repetitively. By these means, the vulnerable phase of nerve regeneration can be utilized better in some situations and a more efficient effect can be achieved.

In a particular form of use, the vanilloid receptor agonist and/or the substance inhibiting nerve regeneration can be used with retarded release. By these means, the vulnerable phase of nerve regeneration can be utilized better in some situations and a more efficient effect can be achieved. The side effects of the two substances can be reduced decisively in this manner locally and systemically.

It has proven to be advantageous if the joint, which is to be treated, is cooled before the application of the agent in order to reduce pain. By so doing, less pain is developed when the substance mixture is injected or applied, since the vanilloid-sensitive ion channels open up more slowly at lower temperatures.

When the inventive agent is used, one or more local anesthetics may be used in addition, either simultaneously with the agent or before the use of the agent. By these means, it is achieved that the injection or administration is not painful. The local anesthetic may be injected at the same place as the agent or remote therefrom. Local pain during the injection can be reduced by these means.

In the following, the invention is implemented in greater detail by means of numerous examples.

### EXAMPLE 1

Under the optionally simultaneous (image converter, CT, sonography, MRI, etc.) or subsequent (x-ray, CT, MRI, sonography, arthroscopy, etc.) imaging control, the therapist brought an injection needle into the joint space of a knee joint and injected 9 mL of a 500 nmolar solution (approximately 0.0001 mg) of resiniferatoxin with 0.3 mg of noscapin into the intracapsular space. The patient noted a clear alleviation of his symptoms already 14 hours after the intervention. This alleviation lasted for more than 6 months.

### EXAMPLE 2

Under the optionally simultaneous (image converter, CT, sonography, MRI, arthroscopy, etc.) or subsequent (x-ray, CT, MRI, sonography, etc.) imaging control, the therapist brought an injection needle into the joint space of a knee joint and injected 9 mL of a 500 nmolar solution (approximately 0.00003 mg) of resiniferatoxin with 0.03 mg of Vincristin into the intracapsular space. The patient noted a clear alleviation of his symptoms already a few days after the intervention. This alleviation lasted for more than 6 months.

### EXAMPLE 3

Under the optionally simultaneous (image converter, CT, sonography, MRI, etc.) or subsequent (x-ray, CT, MRI, sonography, arthroscopy, etc.) imaging control, the therapist brought an injection needle into the joint space of a knee joint and injected 9 mL of a 500 nmolar solution (approximately 0.00003 mg) of resiniferatoxin with 0.03 mg of Vincristin and 1% (approximately 90 mg) of hyaluronic acid into the intracapsular space. The patient noted a clear alleviation of his symptoms already a few days after the intervention. This alleviation lasted for more than 6 months.

### EXAMPLE 4

Under the optionally simultaneous (image converter, CT, sonography, MRI, etc.) or subsequent (x-ray, CT, MRI, sonography, arthroscopy, etc.) imaging control, the therapist brought an injection needle into the joint space of a joint and injected 9 mL of a 500 nmolar solution (approximately 0.00003 mg) of resiniferatoxin with 1% chondroiten sulfate into the intracapsular space. At the same time, the patient perorally took 1 mg of colchicine initially and then 0.5 mg every hour for 12 to 24 hours. Alternatively or in addition to the colchicine, the patient may also take, for example, 50 mg of noscapin 2×1 as syrup or tablet or a comparable substance. The patient noted a clear alleviation of his symptoms already a few days after the intervention. This alleviation lasted for more than 6 months.

### EXAMPLE 5

Under the optionally simultaneous (image converter, CT, sonography, MRI, etc.) or subsequent (x-ray, CT, MRI, sonography, arthroscopy, etc.) imaging control, the therapist brought an injection needle into the joint space of a knee joint and injected 9 mL of a 500 nmolar solution (approximately 0.00003 mg) of resiniferatoxin and 1% of hyaluronic acid into the intracapsular space. At the same time, the patient received 0.5 mg/mL of Vincristin for 24 hours. The patient noted a clear alleviation of his symptoms already a few days after the intervention. This alleviation lasted for more than 6 months.

### EXAMPLE 6

The injected solution corresponded to that of Example 1 with the difference that, for the imaging method to be used, 5 mL of a visible contrasting agent (lopamidol) was added at a concentration of 50 g/100 mL. After the injection, this contrasting agent spread out within the joint capsule and documented the position of the injection needle and the distribution of the substance combination in the joint capsule. The injected mixed solution, containing 500 nmolar (approximately 0.0001 mg) with 0.3 g of noscapin, was drawn off again 30 minutes after the injection. It could, however, also be drawn off after a different, defined, substance-dependent time of action or not be drawn off at all. The patient noted a clear alleviation of his symptoms already 15 hours after the intervention. This alleviation lasted for more than 8 months.

### EXAMPLE 7

The therapist placed a thin infusion catheter, similar to an epidural catheter, into the affected joint and, with a perfuser, injected a mixture of 500 nmolar (approximately 0.0001 mg) of resiniferatoxin with 0.3 mg of vinorelbin into the affected joint at a rate of 1-10 mL/h for 12 hours. Optionally, he also placed a drainage catheter with an optionally defined drainage resistance (such as 20 mm Hg), in order to achieve a liquid turnover. With this method, the therapist achieved a uniform infiltration of the painful joint, without large concentration peaks. Moreover, it was possible to define the period of action better. During subsequent arthroscopies after 1, 2, 7, 14 and 28 days, it was possible to show that only a very little inflamed tissue was present. The patient noted a clear alleviation of his symptoms already 12 hours after the intervention. This alleviation lasted for more than one year.

### EXAMPLE 8

After a knee joint prosthesis had been implanted, the therapist injected 9 mL of a mixture of 500 nmolar (approximately 0.0001 mg) of resiniferatoxin with 0.3 mg of Vincristin into the joint capsule, which had been closed off once again and into the area of surgery. It was possible to minimize postoperative pain by these means.

### EXAMPLE 9

After a hip joint had been implanted, the therapist injected 30 mL of a mixture of 500 nmolar (approximately 0.00003 mg) of resiniferatoxin with 0.01 mg of Vincristin into the periprosthetic region without a capsule. It was possible to minimize postoperative pain by these means.

### EXAMPLE 10

A solution of 2 μmolar (approximately 0.0004 mg) of resiniferatoxin with 1.5 mg of dicoumarol and 0.1 mg of Taxol was injected into the (neo)-capsule about the prosthesis of a patient with a painful, septic loosening of a total hip endoprosthesis. Subsequently, the patient experienced a permanent (more than one year) alleviation of pain within a few (6-12) hours. In addition, the infection about the prosthesis was brought very much under control by the diffusion of the analgesic (which also had antiseptic activity) along the shaft of the prosthesis and about the socket and, in some cases, was even eliminated completely. Optionally, this treatment may be supported with systemically administered antibiotics (such as 450 mg of Rifampicin, 750 mg of ciproflaxacin). It was possible to show radiologically that the bone substance had consolidated about the prosthesis.

### EXAMPLE 11

Under the optionally simultaneous (image converter, CT, sonography, MRI, etc.) or subsequent (x-ray, CT, MRI, sonography, arthroscopy, etc.) imaging control, the therapist brought an injection needle into the joint space of a knee joint and, after administering 5 mL of 2% lidocaine previously as a local anesthetic, injected 9 mL of a solution of 0.01 mg Olvanil with 0.03 mg of Vincristin in a physiological salt solution into the intracapsular space. The patient noted a clear alleviation of his symptoms already a few minutes after the intervention. This alleviation lasted for more than 6 months.

### EXAMPLE 12

Under the optionally simultaneous (image converter, CT, sonography, MRI, etc.) or subsequent (x-ray, CT, MRI, sonography, arthroscopy, etc.) imaging control, the therapist, after optional local anesthesia, brought an injection needle or a catheter into the bladder of the patient and injected 100 mL of a 500 nmolar solution of resiniferatoxin with 0.03 mg of Vincristin, optionally with 10% ethanol. After a period of action of 30 minutes, the solution was drawn off once again.

The patient noted a clear alleviation of his symptoms already a few minutes after the intervention. This alleviation lasted for more than 6 months.

### EXAMPLE 13

The therapist injects 10 mL of a solution of 500 nmolar (approximately 0.0001 mg) resiniferatoxin with 0.3 mg of Nescapin about the Plexus Brachialis or about a different nerve of a patient with shoulder/arm symptoms such as the n. Suprascapularis after optional local anesthesia or under an optional general anesthesia. Already a few minutes after the intervention, the patient noted a clear alleviation of his symptoms, which lasted for more than 6 months.

### EXAMPLE 14

The one mixture of 10 mL of a solution of 500 nmolar (approximately 0.0001 mg) of resiniferatoxin with 0.3 mg of colchicine in a physiological salt solution was injected into the joint of a patient with a painful capsulitis of joints (such as a frozen shoulder or an arthrofibrosis of the knee joint. Once again, it was possible to image and check the distribution of the substance by adding an appropriate contrasting agent. Optionally, a substance with antiphlogistic activity was admixed. A few minutes after the injection, the pain was alleviated permanently, so that the patient regained the mobility, lost due to the capsulitis, by undergoing physiotherapy. For this application, only a temporary analgesia (2-3 weeks) is sometimes required, so that the concentration of the newer toxic substance, if anything, can be kept low here.

### EXAMPLE 15

The therapist injects 500 nmolar solution (approximately 0.00003 mg) of resiniferatoxin with 0.5 mg of doxorubicin into the painful region (attachment of the m. extensor carpi radialis brevis) of a patient with Epicondylitis radialis (tennis elbow), after the elbow previously had optionally been desensitized locally or remotely with a local anesthetic.

### EXAMPLE 16

The joint of a patient with painful capsulitis of joints was injected with 9 mL of a mixture of 500 nmolar (approximately 0.0001 mg) of resiniferatoxin with 0.3 mg of Taxol in a physiological salt solution. The pain was alleviated permanently a few minutes after the injection, so that the patient recovered the mobility, lost due to the capsulitis, by undergoing physiotherapy.

### EXAMPLE 17

After a prior optional local analgesia with 1 mL of 2% lidocaine, the therapist injected 5 mL of a 500 nmolar solution (approximately 0.00005 mg) of resiniferatoxin with 500 mg of griseofulvin, buffered to a pH of 8.0, into a chronically inflamed Bursa trochanterica above the Trochanter major of the hip. Due to the use of a higher pH, the effect set in somewhat more slowly and the patient had fewer symptoms during the injection.

Within 60 minutes, the symptoms of the patient disappeared and the patient remained asymptomatic at this place for several years.

### EXAMPLE 18

A conventional, commercial capsaicin plaster or a corresponding capsaicin cream or a similar cream or ointment was used topically daily on a patient with knee pain. At the same time, the patient took noscapin orally. The noscapin intensified the effect of the ointment massively and the pain, suffered by the patient, was ameliorated for a significantly longer time than it would have been without noscapin.

### EXAMPLE 19

A conventional, commercial capsaicin plaster or a corresponding capsaicin cream or a similar cream or ointment was used topically daily on a patient with back pain. At the same time, the patient took dicoumarol or noscapin orally. The noscapin intensified the effect of the ointment massively and the pain, suffered by the patient, was ameliorated for a significantly longer time than it would have been without dicoumarol or noscapin.

### EXAMPLE 20

A conventional, commercial capsaicin plaster or a corresponding capsaicin cream or a similar cream or ointment was used topically daily on a patient with knee pain. At the same time, the patient took dicoumarol or noscapin orally. The noscapin intensified the effect of the ointment massively and the pain, suffered by the patient, was ameliorated for a significantly longer time than it would have been without noscapin.

### EXAMPLE 21

The therapist injected 0.9 mL of a solution consisting of 500 nmolar (approximately 0.00001 mg) resiniferatoxin with 0.03 mg of Vincristin and optionally 1% of hyaluronic acid and/or a local anesthetic, as well as 5% contrasting agent in a physiological salt solution as solvent into a painful, arthrotic finger joint. After about 15 minutes, the symptoms of the patient disappeared for several months. It was possible to document the correct position of the injection needle by means of the contrasting agent.

### EXAMPLE 22

The therapist injected 10 mL of a solution of 500 nmolar (approximately 0.0001 mg) of resiniferatoxin with 0.3 mg of noscapin about the Nervus obturatorius or about a different nerve of a patient with hip pain after optional local analgesia or under optional general anesthesia. The patient noted a distinct amelioration of his symptoms already a few minutes after the intervention. This amelioration lasted for more than 6 months.

### EXAMPLE 23

The therapist injected 10 mL of a solution of 500 nmolar (approximately 0.0001 mg) of resiniferatoxin with 0.3 mg of an noscapin into the pleural cavity of a patient with pleuritis after optional local analgesia or under optional general anesthesia. The patient noted a distinct amelioration of his symptoms already a few minutes after the intervention. This amelioration lasted for more than 6 months.

### EXAMPLE 24

The therapist injected 10 mL of a solution of 500 nmolar (approximately 0.0001 mg) of resiniferatoxin with 0.3 mg of an noscapin into the intervertebral disk of a patient with back pain after optional local analgesia or under optional general anesthesia. The patient noted a distinct amelioration of his symptoms already a few minutes after the intervention. This amelioration lasted for more than 6 months.

1.-60. (canceled)

61. Mixture of a vanilloid receptor agonist with a substance, which inhibits nerve regeneration and is selected from the following groups of substances:
a) vinca alkaloids, preferably Vincristin, Vincristin sulfate, Vinorelbin or Vinflunin;
b) Ansamitocin P-3 (Maytansinoid), Phomopsin A, Dolastatin 10, Ustiloxines, Arenastation A, tricyclic pyrones (such as 3-pyridyl benzopyran), Rhizoxin and analogs;
c) colchicine and colchicine-like substances;
d) podophyllotoxins, combretastasins and nocodazoles
e) coumarins or dicoumarols;
f) quinolones, especially Ciprofloxacin, Cinoxacin, Enoxacin, Fleroxacin;
g) sulfonamides and
h) flavonoids.

62. The mixture of claim 61, wherein the quinolones are, in particular, Ciprofloxacin, Cinoxacin, Enoxacin and Fleroxacin.

63. The mixture of claim 61, wherein the flavonoids are, in particular, quercetin, indolyl oxazoline derivatives, pyrimidinyl pyrazolates and analogs.

64. The mixture of claim 61, wherein the vanilloid receptor agonist is an agonist for type 1 vanilloid receptor (TRPV1).

65. The mixture of claim 61, wherein the vanilloid receptor agonist is selected from the following substances: resinifera compounds, of which, in particular, the resiniferatoxin (RTX), olvanil, capsiate, civamide, SDZ-249-665, DA-5016, arvanil, scutigeral, isovelleral, phorbol 12,13-didecanoate 20 homovanillate, phorbol 12,13-dinonanoates 20-homovanillates, tinyatoxin and comparable substances, as well as analogs, derivatives and salts of the compounds mentioned above.

66. The mixture of claim 61, wherein the vanilloid receptor agonist is a vanilloid, particularly from the group of trans-8-methyl-N-vanillyl-6-nonenamides, N-vanillyl-nonanamides, beta-aminoethyl-substituted phenylalkanamides, methylene substituted N-phenylmethylalkanamides, N-((substituted phenyl)methyl)-cis-monounsaturated alkenamides, beta-aminoethyl-substituted phenyl compounds, N-((substituted phenyl)methyl) diunsaturated amides, N-oleoyl dopamine, a capsaicin analog, such as transcapsaicin, dihydrocapsaicin, cis-capsaicin, as well as analogs, derivatives and salts of the aforementioned compounds.

67. The mixture of claim 61, wherein the mixture additionally contains a local anesthetic.

68. The mixture of claim 61, wherein the mixture additionally contains an x-ray contrasting agent, preferably in the form of gadolinium-containing, iodine-containing or barium-containing substances.

69. The mixture of claim 61, wherein the mixture additionally contains a steroid.

70. The mixture of claim 61, wherein the mixture additionally contains a vasoconstrictor, preferably adrenaline, noradrenaline, phenylephrine or omipressin.

71. The mixture of claim 61, wherein the mixture is dissolved in a solvent, which is compatible with the body, preferably a pharmacologically acceptable vehicle, particularly from the group of sodium chloride injection solution, Ringer's injection solution, isotonic dextrose, sterile water dextrose solution, lactated Ringer's injection solution, distilled water or mixtures thereof.

72. The mixture of claim 61, wherein the mixture additionally contains a permeation promoter, preferably dimethyl sulfoxide, ethoxylated ethylene diglycol, ethanol, phosphatidyl cholines, propylene glycol dipelargonates (DPPG), or glycosylated ethoxylated glycerides.

73. The mixture of claim 61, wherein the mixture additionally contains a calcium salt.

74. The mixture of claim 73, wherein a calcium ion concentration is greater than 2 mmolar and preferably greater than 4 mmolar.

75. The mixture of claim 73, wherein the concentration of salts and islands, dissolved in the solvent, is higher than that in a physiologically normal solution, such as a Ringer lactate solution.

76. The mixture of claim 61, wherein the mixture additionally contains a glycosaminoglycan(chondroitin sulfate), its derivatives or salts.

77. The mixture of claim 76, wherein the glycosaminoglycan constitutes 0.5% to 10% and preferably 1.0% to 3.0% of the total mixture.

78. The mixture of claim 61, wherein the mixture contains additionally a hyaluronic acid, its derivatives or salts.

79. The mixture of claim 78, wherein the hyaluronic acid constitutes 0.1% to 10% and preferably 0.5% to 3% of the total mixture.

80. The mixture of claim 61, wherein the mixture is dissolved in a buffer solution having a pH higher than 7.6 and preferably higher than 8.5.

81. The mixture of claim 61, wherein the mixture is dissolved in a buffer solution having a pH lower than 7.2 and preferably lower than 6.5.

82. The mixture of claim 61, wherein the mixture is formulated in a suitable pharmaceutical preparation, which permits the release of the mixture to be retarded.

83. The mixture of claim 61, wherein the mixture contains a combination of several substances, which inhibit nerve regeneration.

84. The mixture of claim 61, wherein the mixture contains a combination of several vanilloid receptor agonists.

85. Use of the mixture of claim 61 for producing an agent for the treatment of sensations, which are passed on by nerves carrying vanilloid receptors.

86. The use of claim 85 for producing an agent for the topical treatment of sensations, which are passed on by nerves carrying vanilloid receptors.

87. The use of claim 85, wherein the agent is intended to treat the following indications:
a) Wound pain after surgery in the form of a flushing solution for intraoperative application for open or arthroscopic or endoscopic surgery, including liposuction;
b) Joint pain by intraarticular injection in the case of
arthrosis
rheumatoid arthritis
infectious arthritis
chondrocalcinosis
ligamentary damage
meniscus lesion
cartilage damage
synovitis
arthrofibrosis
Sudeck's disease
necrosis of portions of a joint
neuropathic joint pain;
c) Bone pain after bone surgery by application on the bone after iliac crest osteotomy or Hallux-Valgus correction;
d) Bone pain by injection into the bone in the case of necrosis of the head of the femur or into the body of a vertebra in the case of osteochondrosis;
e) Joint stiffness, especially in the case of arthrofibrosis or a frozen shoulder;
f) Muscle pain due to intramuscular injection, especially if there is a tear in muscle fibers, if there is pain after muscular exertion or in the case of spastic diseases;
g) Painful meniscus if there is degeneration of or a tear in the meniscus;
h) Treatment of back pain by injection into the intervertebral disk in the case of the degeneration of or a tear in the intervertebral disk;
i) Painful nerves, especially trigeminus neuralgia, neurinoma, Morton neurinoma, phantom pain or scar neuroma;
j) Toothache, especially in the case of dental caries, all forms of tooth ache, before, during or after tooth extraction, before, during or after a tooth implant, applied topically in the case of parodontitis, or applied topically in the case of an exposed neck of a tooth;
k) Pleuritic complaints;
l) Intestinal complaints, especially in the case of ulcerous colitis, Crohn's disease, anal fissures or hemorrhoids.

88. The use of claim 85, wherein the vanilloid receptor agonist locally has a concentration or dosage, which is equivalent to the following parameters:
a) In the case of resiniferatoxin as vanilloid receptor agonists, a concentration of 5 nmolar to 600 μmolar and preferably of 100 to 5000 nmolar or a dose of 1 ng to 15 mg/kg of body weight and preferably of 10 ng to 50 pg/kg of body weight.
b) In the case of capsaicinoids as vanilloid receptor agonist, a concentration of 0.05% to 10% by weight of the total mixture or a dose of 0.1 to 200 mg/kg of body weight.

89. The use of claim 85, wherein the substance, inhibiting nerve regeneration, is used at a dosage of 0.0001 mg to 50 mg for intra-articular application.

90. The use of claim 89, wherein the dosage is 0.001 mg to 1 mg.

91. The use of claim 85, wherein the substance, inhibiting regeneration of nerves, is used for application per os in one dosage or in repeated dosages initially of 0.001 to 600 mg.

92. The use of claim 85, wherein the agent is injected locally in a suitable solvent, which is compatible with the body, into the pain-affected tissue structure of the patient or administered dropwise topically onto the surgical wound or applied at a peripheral nerve or ganglion or applied suitably transcutaneously.

93. Method for the treatment of joint pain, wherein the agent of claim 61 is injected locally into the intracapsular region or into the joint capsule of a joint affected by pain.

94. The method of claim 93, wherein the agent of claim 61 is dissolved in a solvent, which is compatible with the body and a volume of 0.1 to 150 mL of the solution is injected locally into the intracapsular region or into the joint capsule of the joint affected by pain.

95. The method of claim 93, wherein the nociceptive nerve fibers are made insensitive to pain for at least 14 days and preferably for at least 8 weeks.

96. The method of claim 93, wherein the agent is used at such a concentration, that neurolysis takes place.

97. The method of claim 93, wherein the agent is used locally, regionally, systemically (intravenous, peroral, subcutaneous, intramuscular, etc.) or topically on the skin or mucous membranes.

98. The method of claim 93, wherein the vanilloid receptor agonist and the substance, inhibiting regeneration of nerves, are used simultaneously.

99. The method of claim 93, wherein the vanilloid receptor agonist is used first, after which the substance, inhibiting nerve regeneration, is used.

100. The method of claim 93, wherein the substance, inhibiting nerve regeneration, is used first, after which the vanilloid receptor agonist is used.

101. The method of claim 93, wherein the vanilloid receptor agonist and/or the substance, inhibiting nerve regeneration, are used repetitively.

102. The method of claim 93, wherein the vanilloid receptor agonist and/or the substance, inhibiting nerve regeneration, are used with retarded release.

103. The method of claim 93, wherein the joint, which is to be treated, is cooled before the agent for alleviating the pain is applied.

104. The method of claim 93, wherein a local anesthetic is used additionally, either simultaneously with the agent or before the agent is used.

105. The method of claim 104, wherein the local anesthetic is injected at the same place as the agent or remote from this place.
